(19) United States
(12) Patent Application Publication
(10) Pub. No.: US 2016/0113559 A1
(43) Pub. Date: Apr. 28, 2016

(54) Interface For Displaying Temporal Blood Oxygen Levels

(71) Applicant: DRAEGER MEDICAL SYSTEMS, INC., Telford, PA (US)

(72) Inventors: Robert Michael Lynch, Holland, PA (US); Harald Kneuer, Klingberg (DE); Merouane Djerbal, Newtown, PA (US); Ali Pourrad, Telford, PA (US); Steffen Schmitt, Tremsbuettel (DE)

(21) Appl. No.: 14/778,010

(22) PCT Filed: Mar. 25, 2013
(86) PCT No.: PCT/US2013/033736

§ 371 Date: Sep. 17, 2015

(51) Int. Cl. A61B 5/145; A61B 5/00; A61B 5/0205

(52) U.S. Cl. CPC A61B 5/14542; A61B 5/0205; A61B 5/7278; A61B 5/0004; A61B 5/742; A61B 5/748; A61B 5/746; A61B 5/7246; A61B 2503/045; A61B 2560/0475

(57) ABSTRACT

A blood oxygen graph (200) that specifies blood oxygen level ranges varying over a span of time is displayed within a graphical user interface. Data from at least one physiological sensor is received that includes a current blood oxygen level (230) of a patient and a time point (260) associated with the measurement. Using the received data, the current blood oxygen level for the patient overlaid on the blood oxygen graph at the time point is displayed in the graphical user interface. Related apparatus, systems, techniques, and articles are also described.

### TECHNICAL FIELD

The subject matter described herein relates to displaying physiological parameters such as measured blood oxygen levels for neonatal care.

### BACKGROUND

The APGAR score is a simple and repeatable method to assess the health of newborn children (i.e., neonates) immediately after birth. A physician determines the APGAR score by evaluating the neonate on five criteria. The physician judges each criterion on a scale from zero to two, and then sums the five values. The resulting APGAR score ranges from zero to ten. The five criteria can summarize as appearance, pulse, grimace, activity, and respiration (APGAR). A physician generally performs the APGAR test at one and five minutes after birth, and may repeat the test later if the score is and remains low. Scores seven and above are normal, scores four to six are low and three and scores below three are critically low.

A low score on the one-minute test may show that the neonate requires medical attention but is not necessarily an indication that there will be long-term problems, particularly if there is an improvement by the stage of the five-minute test. If the APGAR score remains below three such as at 10, 15, or 30 minutes, there is a risk that the child will suffer longer-term neurological damage. The purpose of the APGAR test is to determine quickly whether a newborn needs immediate medical care. An APGAR timer, in its simplest form, is simply a stop watch or egg timer that is begun at the time of birth to remind a physician to measure the neonates APGAR score at 1, 5, and 10 minutes from birth.

### SUMMARY

In one aspect, a blood oxygen graph that specifies blood oxygen level ranges varying over a span of time is displayed within a graphical user interface. Data from at least one physiological sensor is received that includes a current blood oxygen level of a patient and a time point associated with the measurement. Using the received data, the current blood oxygen level for the patient overlaid on the blood oxygen graph at the time point is displayed in the graphical user interface.

In another aspect, data characterizing a start of an APGAR timer is received. In response to the received data and based on time, a predetermined blood oxygen threshold value is incremented to create a dynamic blood oxygen threshold value that varies over time. The dynamic blood oxygen threshold value is compared to a measured blood oxygen level. Data characterizing the comparison is provided.

In yet another aspect, a system includes a physiological sensor measuring a blood oxygen level of a patient, a computing system receiving data from the physiological sensor, and a display coupled to the computing system. A blood oxygen graph that specifies blood oxygen level ranges varying over a span of time is displayed within a graphical user interface. Data from at least one physiological sensor comprising a current blood oxygen level of a patient and a time point associated with the measurement is received. Using the received data, the current blood oxygen level for the patient overlaid on the blood oxygen graph at the time point is displayed within the graphical user interface.

In yet another aspect, a system includes a computing system coupled to an APGAR timer. Data is received characterizing a start of the APGAR timer. In response to the received data and based on time, a predetermined blood oxygen threshold value is incremented to create a dynamic blood oxygen threshold value that varies over time. The dynamic blood oxygen threshold value is compared to a measured blood oxygen level. Data characterizing the comparison is provided.

One or more of the following features can be included. For example, the one or more blood oxygen level ranges can include a lower threshold that varies over the span of time. The one or more blood oxygen level ranges can include an upper threshold. The one or more blood oxygen level ranges can indicate at least one of the following: a normal range, a caution range, and a critical range. One or more past blood oxygen levels for the patient overlaid on the blood oxygen graph at past time points can be displayed. The span of time can be dynamic based at least on the received time point. Data can be received from the at least one physiological sensor characterizing a measure of confidence in the blood oxygen level of the patient and, using the received data, the measure of confidence in the blood oxygen level of the patient overlaid on the blood oxygen graph at the time point can be displayed in the graphical user interface. Data can be received from at least one physiological sensor comprising an additional parameter of the patient and, using the received data, the additional parameter of the patient overlaid on the blood oxygen graph can be displayed.

The blood oxygen graph can be selected from a plurality of predetermined blood oxygen graphs. The selection of the blood oxygen graph can be based on a physiological parameter of the patient. The blood oxygen graph can be generated based on a physiological parameter of the patient. The physiological parameter of the patient can include at least one of heart rate, temperature, birth weight, gestational age, and provided oxygen level (FiO2). The patient can be a neonate having been born within twenty minutes of the time point. The at least one physiological sensor includes a pulse oximeter. The displaying can occur on a display device remote from the physiological sensor. The displaying can occur on a display integral with one or more of the following: smart phone, tablet computer, bedside patient monitor, and warmer.

The point in time can be measured from a start of an appearance, pulse, grimace, activity, and respiration (APGAR) timer. The time point can be synchronized to an APGAR timer. The blood oxygen ranges can vary based on an APGAR timer. Measured blood oxygen levels and associated time points can be continually received, and the display can be dynamically updated. The continually received measured blood oxygen levels and associated time points can be tracked, and past blood oxygen levels for the patient overlaid on the blood oxygen graph at their associated time points can be displayed. Measured blood oxygen levels can be continually received and continually compared to the dynamic blood oxygen threshold value.

An alarm can be provided when the received blood oxygen level of the patient at the time point is below the lower threshold or above the upper threshold. Providing the alarm can include one or more of the following: flashing a light, displaying predetermined written instructions, providing a predetermined noise, and providing a pre-generated voice message. Providing data characterizing the comparison can include at least one of transmitting, storing, persisting, and displaying. Providing data characterizing the comparison can include displaying, using a graphical user interface, the dynamic blood oxygen threshold and the measured blood oxygen level. Providing data characterizing the comparison can include providing an alarm when the measured blood oxygen level is below the dynamic blood oxygen threshold value. An alarm can be provided when the measured blood oxygen level is out of bounds of the dynamic blood oxygen threshold value. An alarm can be generated when the measured value is out of bounds of the dynamic blood oxygen threshold.

Computer program products are also described that comprise non-transitory computer readable media storing instructions, which when executed by at least one data processors of one or more computing systems, causes at least one data processor to perform operations herein. Similarly, computer systems are also described that may include one or more data processors and a memory coupled to the one or more data processors. The memory may temporarily or permanently store instructions that cause at least one processor to perform one or more of the operations described herein. In addition, methods can be implemented by one or more data processors either within a single computing system or distributed among two or more computing systems.

The subject matter described herein provides many advantages. For example, the current subject matter aids a caregiver during the stabilization phase after birth by providing for a clear unambiguous indication of neonate status. Further, a quantitative comparison of a specific neonate's blood oxygen ranges to healthy ranges is provided. Additionally, the current subject matter enables quick response and medical attention to neonates with abnormal blood oxygen levels immediately after birth and educates health workers that low blood oxygen levels in the recently born is common. Also, the amount and frequency of unnecessary alarms from patient monitoring equipment are reduced.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a process flow diagram illustrating a method for displaying a patient's physiological data such as blood oxygen levels;

FIG. 2 is a drawing illustrating an example blood oxygen graph with a lower threshold and upper threshold, both of which vary over time;

FIG. 3 is a drawing illustrating another example blood oxygen graph with multiple lower thresholds, and multiple upper thresholds;

FIG. 4 is a drawing illustrating another example blood oxygen graph with additional displayed parameters;

FIG. 5 is a drawing illustrating another example blood oxygen graph with displayed measures of blood oxygen level confidence;

FIG. 6 is a drawing illustrating another example blood oxygen graph showing past blood oxygen levels below the lower threshold;

FIG. 7 is a block diagram illustrating a system for displaying a patient's current blood oxygen;

FIG. 8 is a process flow diagram illustrating a method for synchronizing an APGAR timer and a dynamic blood oxygen threshold; and

FIG. 9 is a drawing illustrating an example blood oxygen graph with a lower dynamic blood oxygen threshold that increments in steps.

Like reference symbols in the various drawings indicate like elements.

### DETAILED DESCRIPTION

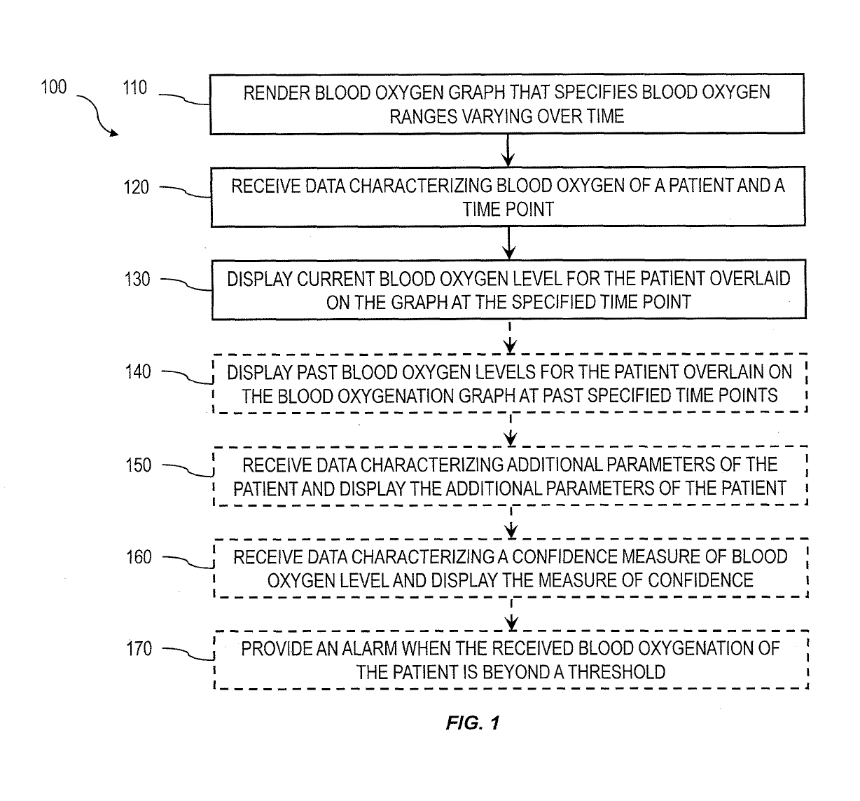

FIG. 1 is a process flow diagram illustrating a method 100 for displaying a patient's physiological data such as blood oxygen levels. At 110, a blood oxygen graph is displayed that specifies blood oxygen ranges varying over time. The displaying can occur within a graphical user interface. At 120, data is received characterizing (and/or including) blood oxygen levels of the patient and a time point. The data can be received from at least one physiological sensor. The time point can be synchronized with a starting of an APGAR timer. At 130, a current blood oxygen level for the patient overlaid on the graph at the time point is displayed. Optionally, at 140, past blood oxygen levels overlaid on the blood oxygen graph at past time points can be displayed.

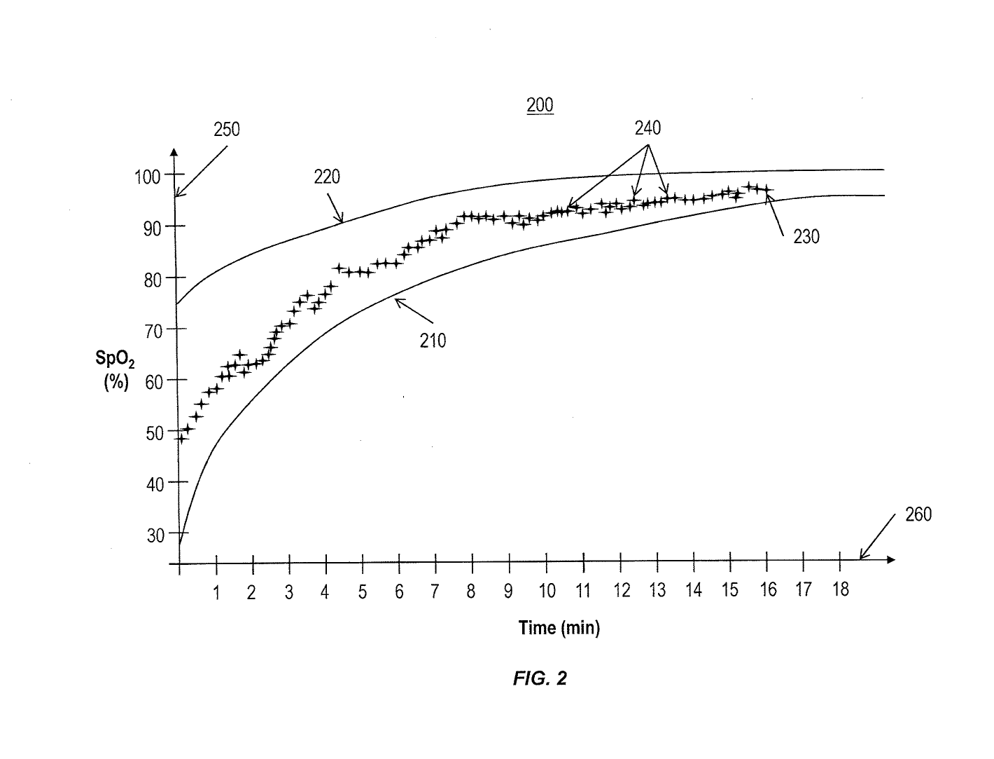

FIG. 2 is a drawing of one implementation of the current subject matter illustrating an example blood oxygen graph 200 with a lower threshold 210 and upper threshold 220, both of which vary over time. Stars indicate blood oxygen levels. The current blood oxygen level is shown at 230 and past blood oxygen levels are shown at least at 240. The vertical axis 250 coordinates are in units of blood oxygen (SpO2) percentage and the horizontal axis 260 coordinates are in units of time (i.e., minutes). In this example, the patient is a neonate, that is, someone recently born. When the patient is a neonate, time can be measured from approximately the moment of birth and/or synchronized to the starting of an APGAR timer. The horizontal axis 260 can dynamically expand. The expansion can be based on the specified time point. In this example, the patient was born less than twenty minutes before the display of the current blood oxygen point 230.

In this example, the lower threshold 210 and upper threshold 220 both monotonically increase over time. The lower threshold 210 and upper threshold 220 variations reflect outer bounds for normal blood oxygen level variations of neonates shortly after birth. Once the upper threshold 220 reaches 100 percent blood oxygen level, the upper threshold 220 can remain constant over time. Each threshold can begin to vary (e.g., increment) in response to a resetting (i.e., starting) of an APGAR timer. Thus, in certain implementations, the thresholds are synchronized to the APGAR timer.

Additionally, in this example, all displayed blood oxygen levels reside above the lower threshold 210 and below the upper threshold 220. The region between the lower threshold 210 and upper threshold 220 can represent a normal, healthy, and/or expected range of blood oxygen levels (at a given time) and can indicate that the patient's blood oxygen level is at a healthy level. Regions below the lower threshold 210 and/or above the upper threshold 220 can represent abnormal, unhealthy, and/or unexpected ranges of blood oxygen levels, and can indicate that the patient is in need of medical care. One or more regions can be shaded or colored (e.g., green, yellow, or red) to improve contrast and readability.

The blood oxygen graph 200 can be selected from a plurality of predetermined blood oxygen graphs. The predetermined blood oxygen graphs can include different threshold values (i.e., different variations over time and different absolute threshold values). The selection can be manual (e.g., by a physician or other person) or can be selected based on a physiological parameter of the patient. The selection can be automatic. The physiological parameter can include at least one of the blood oxygen level, heart rate, temperature, birth weight, and gestational age. Alternatively, the blood oxygen graph can be generated based at least on a physiological parameter of the patient. Predetermined threshold values and variations can be selected. The selection can be manual or automatic and can be based on a physiological parameter.

The measured blood oxygen levels can be continuously received in regular or irregular time increments and the display can be continuously and/or dynamically updated with each received measured blood oxygen level. The display can be updated after receiving several measured blood oxygen levels. In the example shown in FIG. 2, past blood oxygen levels are shown at 240. As the measured blood oxygen levels are being continuously received they can be tracked (e.g., stored or recorded) and the display can be refreshed or the most recently received data can be overlaid on the display. In addition to or as an alternative to tracking the continuously received data, the past blood oxygen levels can be received in addition to the current blood oxygen level. This allows a person viewing the display to ascertain the historical blood oxygen levels of the patient.

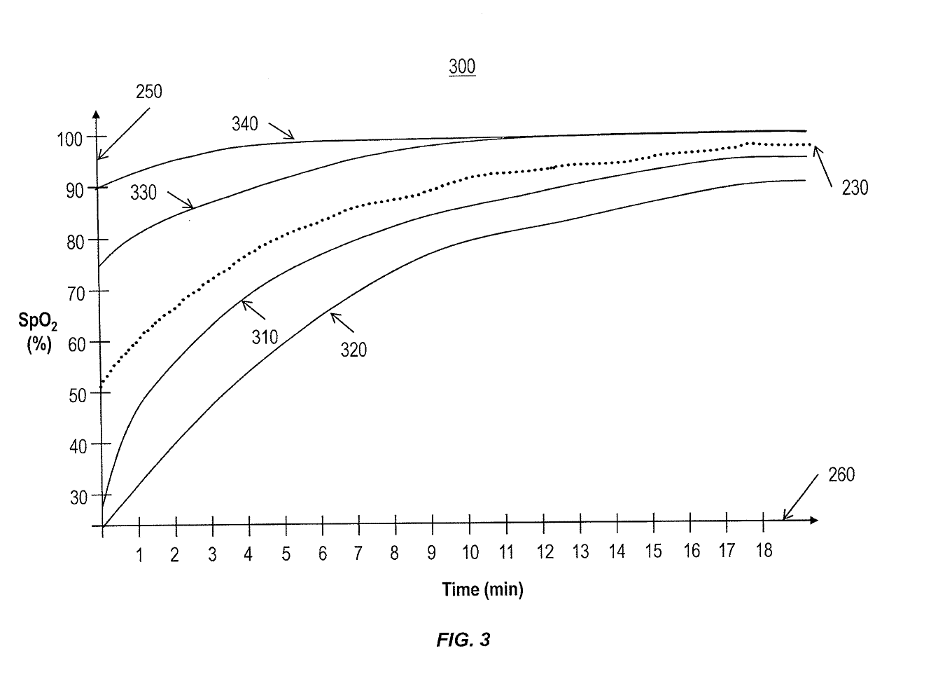

FIG. 3 is a drawing of another implementation of the current subject matter illustrating an example blood oxygen graph 300 with multiple lower thresholds (310 and 320), and multiple upper thresholds (330 and 340). Here each blood oxygen level is shown as a dot. The region between the first lower threshold 310 and the first upper threshold 330 can represent a normal or expected range of blood oxygen levels and can indicate that the patient's blood oxygen level is at a healthy or expected level. The region between the first lower threshold 310 and second lower threshold 320 and/or between the first upper threshold 330 and second upper threshold 340 can represent a caution region. Blood oxygen values displayed in the caution region can indicate that the patient requires medical attention and/or close monitoring. Regions below the second lower threshold 320 and above the second upper threshold 340 can represent critical regions. Blood oxygen values displayed in one of the critical regions can indicate that the patient requires immediate medical care.

Referring again to FIG. 1, optionally, at 150, data characterizing additional parameters of the patient can be received and displayed. The additional parameters can include a patient's heart rate, temperature, gestational age, birth weight, and provided oxygen level (FiO2). The patient's heart rate can be obtained, for example, from the physiological sensor that is measuring the patient's blood oxygen or from another sensor (e.g., electrocardiogram, non-invasive blood pressure, etc.). The patient's heart rate can be received or determined from data characterizing one or more patient physiological parameters.

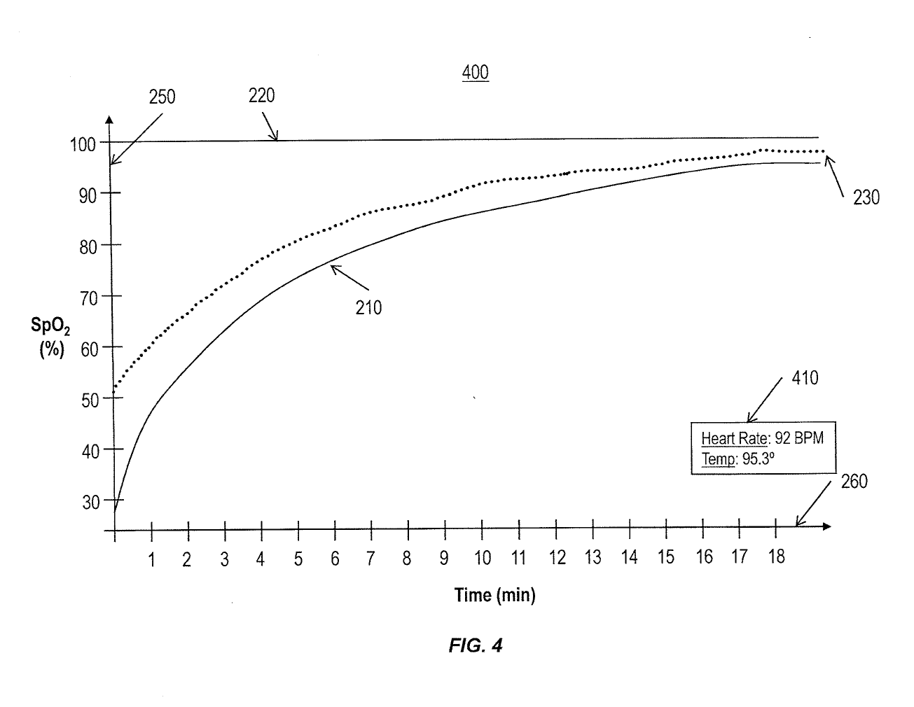

FIG. 4 is a drawing of another implementation of the current subject matter illustrating an example blood oxygen graph 400 with additional displayed parameters 410. Similar to FIG. 2, graph 300 includes a lower threshold 210, upper threshold 220, current blood oxygen level 230, past blood oxygen levels, vertical axis 250, and horizontal axis 260. In this example, the upper threshold 220 is constant at 100 percent while the lower threshold 210 varies over time. The upper threshold 220 need not be shown. The additional parameters are displayed at 410, and can be relevant to determining a patient's overall medical condition. The additional parameters can also be displayed graphically, such as, for example, overlaying FiO2 values on the blood oxygen graph to show present and/or past FiO2 values over time. For FiO2, in some variations, no additional axis scale (x-axis or y-axis) would be required because FiO2 values can be displayed as a percentage. The displayed blood oxygen levels and additional parameters can be stored for later retrieval.

Referring again to FIG. 1, optionally, at 160, data characterizing a confidence measure of the blood oxygen levels can be received and displayed. The confidence measure can be received from the physiological sensor.

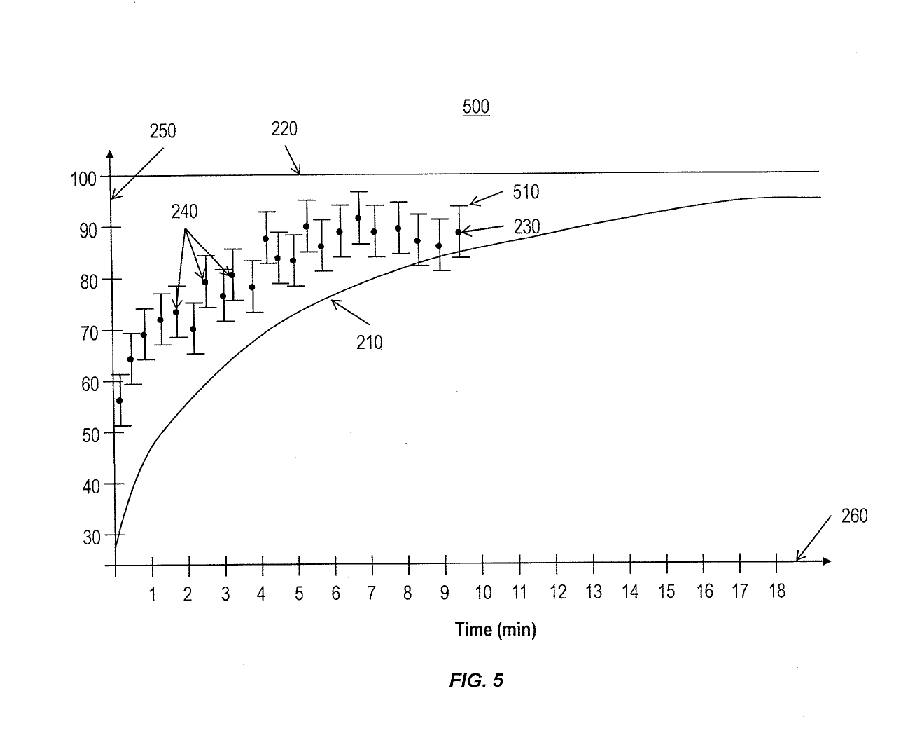

FIG. 5 is a drawing of another implementation of the current subject matter illustrating an example blood oxygen graph 500 with displayed measures of blood oxygen level confidence. In this example, the displayed current blood oxygen level 230 (and displayed past blood oxygen levels 240) has an error bar 510 illustrating the measure of confidence of the physiological parameter. The confidence can include a certainty, likelihood, or probability that the patient's actual blood oxygen level is within the error bar (e.g., a 99% chance that the patient's blood oxygen falls within the bounds of the error bar).

Referring again to FIG. 1, optionally, at 170, an alarm or warning can be provided or generated when the received blood oxygen levels of the patient is out of the bounds of a threshold. For example, if the received blood oxygen level of the patient is below the lower threshold 210 or above the upper threshold 220, a light can flash, predetermined written instructions can display, a predetermined noise can sound, and/or a pre-generated voice message can be provided.

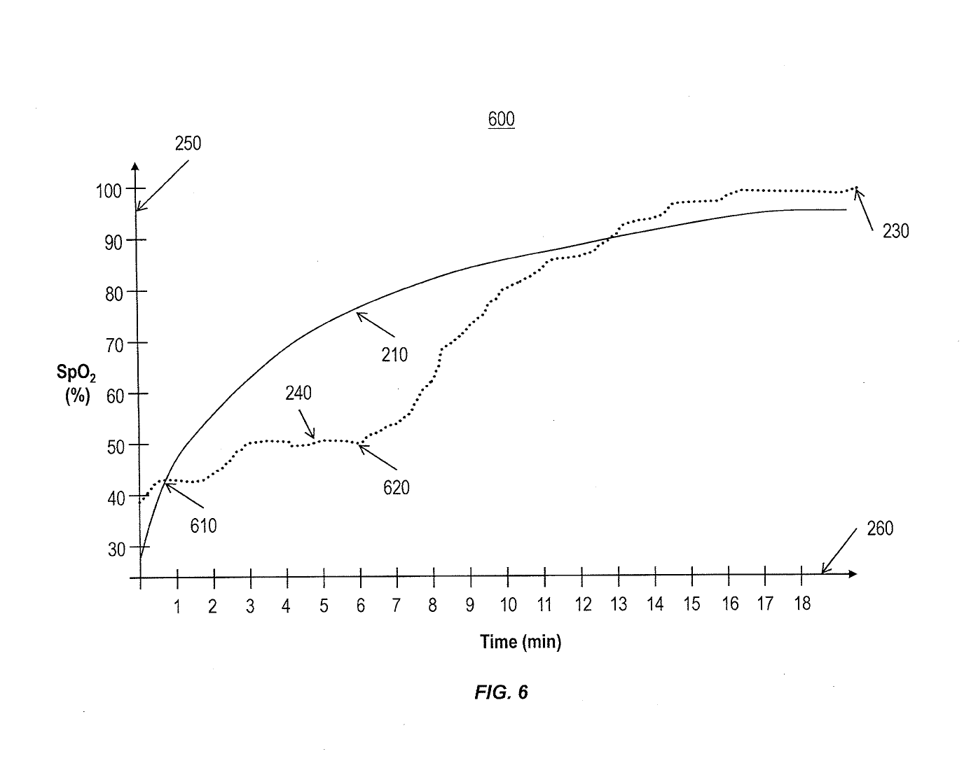

FIG. 6 is a drawing of another implementation of the current subject matter illustrating an example blood oxygen graph 600 showing past blood oxygen levels 240 below the lower threshold 210. In the example shown, after approximately one minute, at 610, the past blood oxygen levels 240 transitions from above the lower threshold 210 to below the lower threshold 210. Optionally, when this occurs, an alarm may sound indicating the patient may require medical attention. In the example shown, after approximately six minutes, at 620, the blood oxygen level increases sharply. This can be the result of resuscitation efforts (e.g., increasing FiO2) of a physician. Resuscitation efforts can include, for example, warming, clearing the patient's airway, stimulation, ventilation corrective steps, incubation, chest compressions, and intravenous epinephrine. The implementation of FIG. 6 does not include an upper threshold.

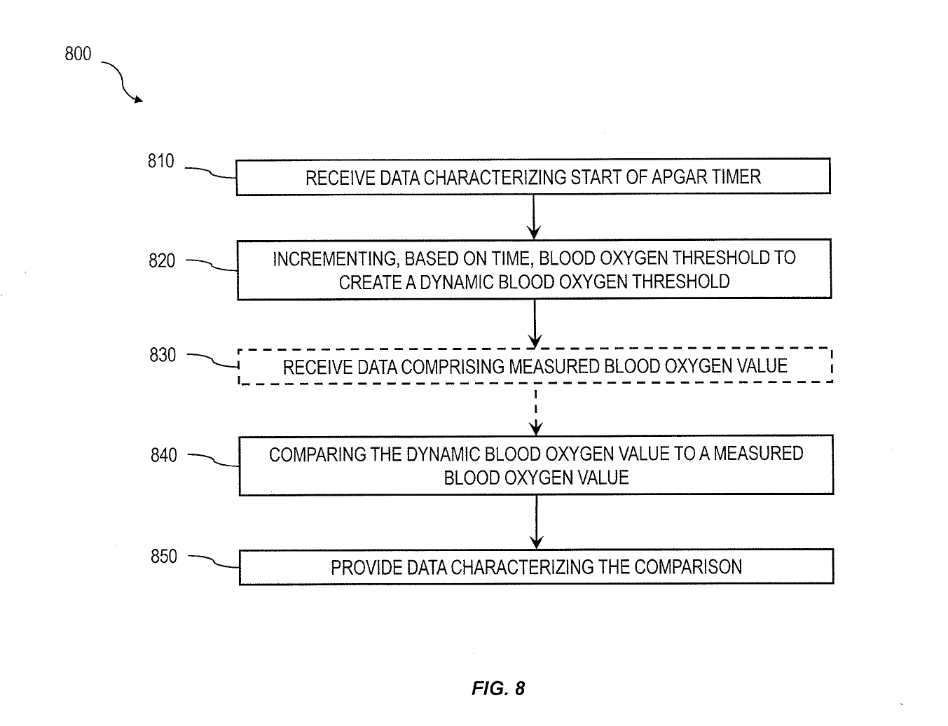

FIG. 8 is a process flow diagram illustrating a method 800 for synchronizing an APGAR timer and a dynamic blood oxygen threshold. Data is received at 810 that characterizes a start of an APGAR timer. The APGAR timer can include, for example, a device dedicated to monitoring time, a software module in a blood oxygen measurement device, a software module in a computing system, or a module in a device that displays measured blood oxygen data. A physician or other health care professional can start the APGAR timer. Typically, a physician starts the APGAR timer approximately at the time of birth of a neonate.

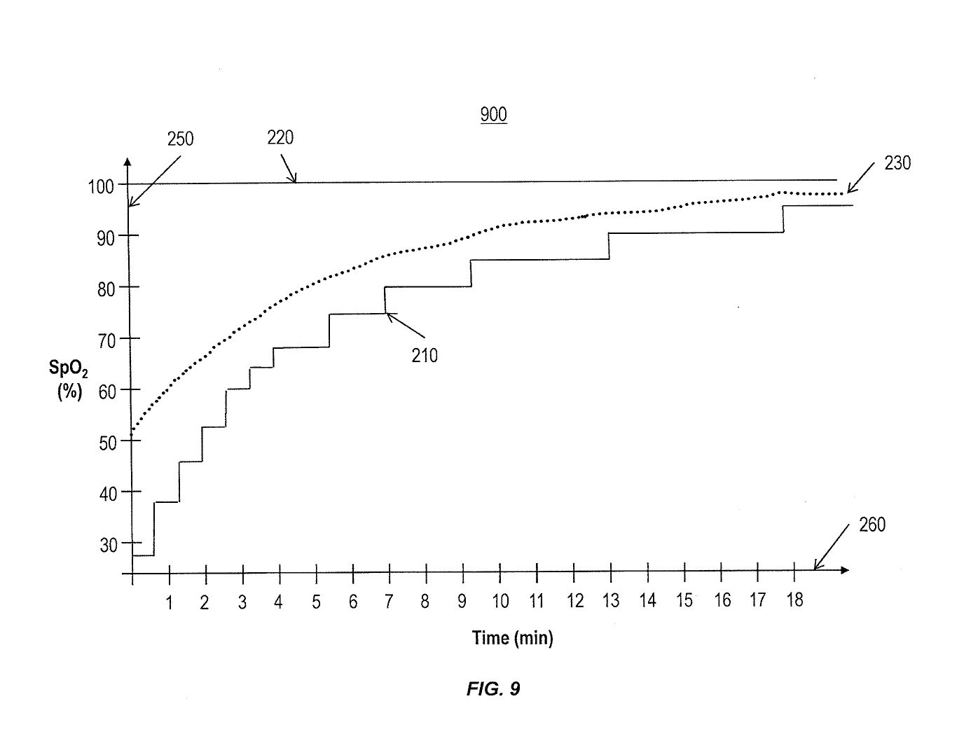

In response to the received data at 820 a blood oxygen threshold is incremented to create a dynamic blood oxygen threshold that varies over a time span. The dynamic blood oxygen threshold can increment based on time. The incrementing can be performed continually to result in dynamic blood oxygen thresholds similar to those illustrated in FIGS. 2-6. The dynamic blood oxygen threshold can increment in steps (e.g., stepwise). FIG. 9 is a drawing of another implementation of the current subject matter illustrating an example blood oxygen graph 900 showing past blood oxygen levels 240 and a lower dynamic blood oxygen threshold 210 that increments in steps. The steps are shown in FIG. 9 as being irregularly spaced in time, however, steps incrementing at regularly spaced intervals, or according to a predetermined or predefined incrementing schedule is also possible. Referring again to FIG. 8, optionally, data can be received at 830, the data comprising a measured blood oxygen value.

The dynamic blood oxygen threshold is compared at 840 to the measured blood oxygen level. The comparison can include directly comparing values (e.g., to show the measured level is greater than, less than, and/or equal to the threshold), and displaying in a graphical user interface the dynamic blood oxygen threshold and the measured blood oxygen level. Additionally, measured blood oxygen levels can be continually received and continually compared to the dynamic blood oxygen threshold value.

Data characterizing the comparison is provided at 850. Providing can include, for example, transmitting, storing, persisting, and displaying. An alarm can be provided. The alarm can be in response to the comparison, such as when the measured blood oxygen level is below (or above) the dynamic threshold value for a given comparison. Providing can include displaying, using a graphical user interface, the dynamic blood oxygen threshold and the measured blood oxygen level.

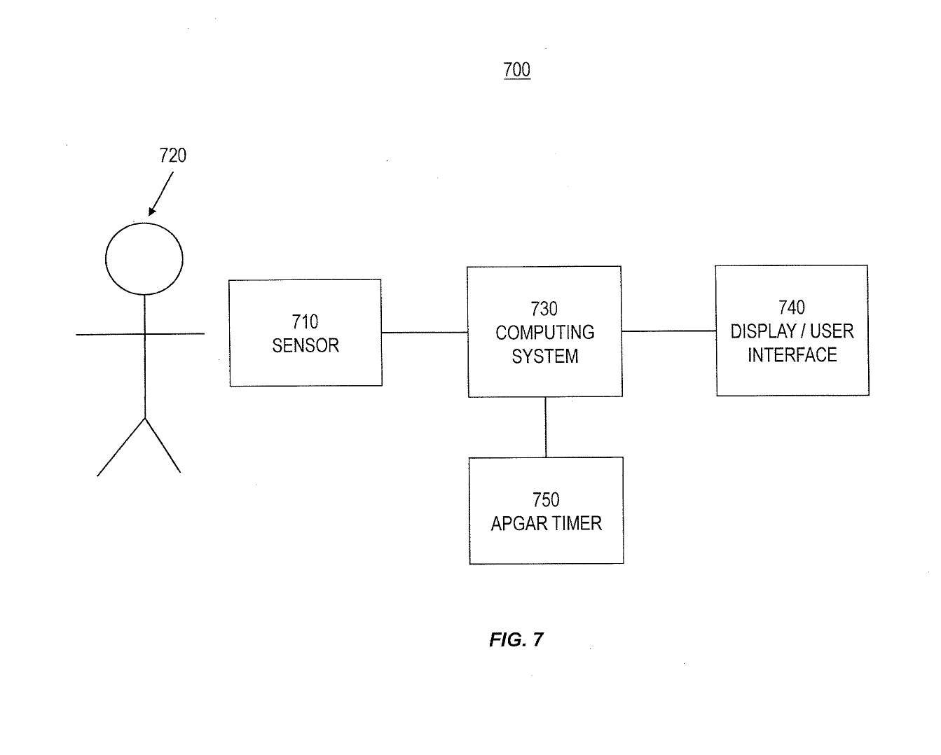

FIG. 7 is a block diagram illustrating a system 700 for displaying a patient's current blood oxygen. A physiological sensor 710 measures at least one physiological parameter of a patient 710. The physiological sensor 710 can include a pulse oximeter sensor. The sensor 710 is coupled to a computing system 730. The coupling can be direct (e.g., over a wire), or indirect (e.g., over a distributed hospital network). The sensor 710 and computing system 730 can be remote from each other. The computing system 730 is also coupled directly or indirectly to a display and/or user interface 740. The computing system 730 can also be coupled to, integrated with, and/or in communication with an APGAR timer 750 to receive a signal or data from the APGAR timer characterizing a start of the APGAR timer and/or an elapsed time from the start of the APGAR timer. Implementations of the computing system 730 and/or display 740 can include a bedside patient monitor, a smart phone, tablet, desktop PC, laptop, newborn baby warmer, APGAR timer, and resuscitation unit.

While implementations shown herein have included between one and four thresholds, any number of thresholds and/or regions are possible. Additionally, the blood oxygen levels can be processed. For example, current and past blood oxygen levels can be smoothed in time (e.g., a sliding average) or low pass filtered to remove high frequency components. The incrementing of the dynamic blood oxygen threshold can be performed by any of the modules in the system such as, for example, the sensor 710, the computing system 730, and the display 740, or by another module.

Various implementations of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor) for displaying information to the user and a keyboard and a pointing device (e.g., a mouse, a trackball, or touchscreen) by which the user may provide input to the computer. Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other.

Although a few variations have been described in detail above, other modifications are possible. For example, the logic flow depicted in the accompanying figures and described herein do not require the particular order shown, or sequential order, to achieve desirable results. Other embodiments may be within the scope of the following claims.

1.-26. (canceled)

27. A method comprising:
receiving data characterizing a start of an APGAR timer;
incrementing, in response to the received data and based on time, a predetermined blood oxygen threshold value to create a dynamic blood oxygen threshold value that varies over time;
comparing the dynamic blood oxygen threshold value to a measured blood oxygen level; and
providing data characterizing the comparison;
wherein at least one of receiving, incrementing, comparing, or providing is performed by at least one data processor forming part of one or more computing systems.

28. The method of claim 27, further comprising:
receiving data comprising the measured blood oxygen level.

29. The method of claim 27 further comprising continually receiving measured blood oxygen levels and continually comparing the dynamic blood oxygen threshold value to the received measured blood oxygen levels.

30. The method of claim 27, wherein providing data characterizing the comparison includes at least one of transmitting, storing, persisting, and displaying.

31. The method of claim 27, wherein providing data characterizing the comparison includes displaying, using a graphical user interface, the dynamic blood oxygen threshold and the measured blood oxygen level.

32. The method of claim 27, wherein providing data characterizing the comparison includes providing an alarm when the measured blood oxygen level is below the dynamic blood oxygen threshold value.

33. A non-transitory computer program product storing instructions, which when executed by at least one data processor of at least one computing system, implement a method comprising:
receiving data characterizing a start of an APGAR timer;
incrementing, in response to the received data and based on time, a predetermined blood oxygen threshold value to create a dynamic blood oxygen threshold value that varies over time;
comparing the dynamic blood oxygen threshold value to a measured blood oxygen level; and
providing data characterizing the comparison;
wherein at least one of receiving, incrementing, comparing, and providing is performed by at least one data processor forming part of one or more computing systems.

34. A system comprising: at least one data processor; and memory storing instructions, which when executed by the at least one data processor, implement a method comprising:
receiving data characterizing a start of an APGAR timer;
incrementing, in response to the received data and based on time, a predetermined blood oxygen threshold value to create a dynamic blood oxygen threshold value that varies over time;
comparing the dynamic blood oxygen threshold value to a measured blood oxygen level; and
providing data characterizing the comparison.

35. A system comprising:
a computing system coupled to an APGAR timer;
the computing system including at least one data processor and memory storing instructions which, when executed by the at least one data processor, causes the at least one data processor to perform operations comprising:
receiving data characterizing a start of the APGAR timer;
incrementing, in response to the received data and based on time, a predetermined blood oxygen threshold value to create a dynamic blood oxygen threshold value that varies over time;
comparing the dynamic blood oxygen threshold value to a measured blood oxygen level; and
providing data characterizing the comparison.

36. The system of claim 35, wherein providing data characterizing the comparison includes providing an alarm when the measured blood oxygen level is out of bounds of the dynamic blood oxygen threshold value.

37. The system of claim 35, the method further comprising generating an alarm when the measured value is out of bounds of the dynamic blood oxygen threshold.

38. The computer program product of claim 33, the method further comprising:
receiving data comprising the measured blood oxygen level.

39. The computer program product of claim 33, the method further comprising continually receiving measured blood oxygen levels and continually comparing the dynamic blood oxygen threshold value to the received measured blood oxygen levels.

40. The computer program product of claim 33, wherein providing data characterizing the comparison includes at least one of transmitting, storing, persisting, and displaying.

41. The computer program product of claim 33, wherein providing data characterizing the comparison includes displaying, using a graphical user interface, the dynamic blood oxygen threshold and the measured blood oxygen level.

42. The computer program product of claim 33, wherein providing data characterizing the comparison includes providing an alarm when the measured blood oxygen level is below the dynamic blood oxygen threshold value.

43. The system of claim 34, the method further comprising:
receiving data comprising the measured blood oxygen level.

44. The system of claim 34, the method further comprising continually receiving measured blood oxygen levels and continually comparing the dynamic blood oxygen threshold value to the received measured blood oxygen levels.

45. The system of claim 34, wherein providing data characterizing the comparison includes at least one of transmitting, storing, persisting, and displaying.

46. The system of claim 34, wherein providing data characterizing the comparison includes displaying, using a graphical user interface, the dynamic blood oxygen threshold and the measured blood oxygen level.
